(19) United States
(12) Patent Application Publication
(10) Pub. No.: US 2014/0350405 A1
(43) Pub. Date: Nov. 27, 2014

(54) SYSTEM AND METHOD FOR IDENTIFYING HIGH RISK PREGNANCIES

(71) Applicant: KONINKLIJKE PHILIPS N.V., EINDHOVEN (NL)

(72) Inventors: Pallavi Vajinepalli, Bangalore (IN); Ranjan Das, Orissa (IN); Celine Firtion, Surat (IN); Rajendra Singh Sisodia, Bhopal (M.P.) (IN); Lalit Gupta, Noida (IN); Ganesan Ramachandran, Bangalore (IN); Ajay Anand, Fishkill, NY (US); John Petruzzello, Carmel, NY (US)

(21) Appl. No.: 14/361,312

(22) PCT Filed: Nov. 26, 2012
(86) PCT No.: PCT/IB2012/056726

§ 371 Date: May 29, 2014

(30) Foreign Application Priority Data
Nov. 30, 2011 (IN) 4125/CHE/2011

(51) Int. Cl. A61B 5/00; A61B 8/06; A61B 8/08

(52) U.S. Cl. CPC A61B 5/4343; A61B 8/488; A61B 8/06; A61B 8/0866
USPC 600/454

(57) ABSTRACT

Method, system and software product for identifying high risk pregnancies comprising the step of generating a spectrogram from ultrasound Doppler signals reflected from the uterine artery and determining the maximum frequency envelope of said spectrogram, and of defining a systolic part and a diastolic part of the maximum frequency envelope and calculating an area ratio under said systolic and diastolic part (AR). This area ratio relates to the blood volume in the uterine artery.

### FIELD OF THE INVENTION

The invention relates to a system and method for identifying high risk pregnancies.

### BACKGROUND OF THE INVENTION

Assessing risks of pregnancy and monitoring high risk pregnancies are daily routine in pregnancy care, so as to help a mother to give birth to a healthy baby. Currently, a way to assess fetal well-being and to classify the risk of a pregnancy is to measure the maternal and fetal blood flow of different blood vessels of mother and fetus using ultrasound Doppler. Risks and complications of pregnancy are reflected by anomalous blood vessels' flows. For example, an abnormal uterine artery flow may reflect high risk pregnancy conditions such as pre-eclampsia, maternal hypertension and intra-uterine growth restriction (IUGR), and may be an indication for prenatal death.

Currently, ultrasound Doppler waveform analyses of specific blood flows of fetus and mother is routinely used to detect and monitor high risk pregnancies. Doppler waveform analysis of the blood flow in the uterine artery is used by obstetricians and sonologists to assess the utero-placental circulation in monitoring and detecting high risk conditions, like hypertensive disorders of pregnancy (Pregnancy induced hypertension (PIH) and Pre-eclampsia) and fetal Intrauterine growth restriction (IUGR).

The Resistive Index (RI), Pulsatility Index (PI) and the Peak Systolic Velocity/End Diastolic Velocity ratio (S/D-ratio) are widely used parameters to quantify the blood flow in the uterine artery. RI, PI, and S/D-ratio are ratios computed from the peak or average systole and diastole blood flow (see for example “Use of uterine artery Doppler ultrasonography to predict pre-eclampsia and intrauterine growth restriction: a systematic review and bivariable meta-analysis”, CMAJ, Mar. 11, 2008).

These parameters are preferred parameters because they can be determined from angle independent measurements (i.e., they do not depend on the angle of incidence between the ultrasound wave and the blood vessel). However, these parameters do not provide any direct estimation of the amount of blood in the vessels.

Although the above mentioned parameters are considered the gold standard as far as current clinical practice goes, they still suffer from low sensitivity in detecting and predicting the above mentioned high risk pregnancy conditions at an early stage. Furthermore, the sensitivity of these parameters has been found to be highly variable in various studies, despite their high specificity in diagnosing high risk pregnancies.

In this respect the sensitivity is defined as the ratio of the number of true positives over the sum of the number of true positives and false negatives.

### SUMMARY OF THE INVENTION

It is therefore an object of the invention to provide a system and method which have an improved sensitivity in detecting high risk pregnancies.

In accordance with a method according to the invention, the method comprises the steps of acquiring ultrasound Doppler signals from the uterine artery; of generating a spectrogram from the acquired ultrasound Doppler signals and determining the maximum frequency envelope of said spectrogram; and of defining a systolic part and a diastolic part of the maximum frequency envelope and calculating an area ratio under said systolic and diastolic part (AR).

According to the invention determining an area ratio (AR) of the systolic part and the diastolic part of the area under a curve representing the maximum frequency envelope of a spectrogram from the acquired ultrasound Doppler signals is proposed. This parameter is an indirect indication of the blood volume in the uterine artery.

The inventors have recognized that measuring blood volume in the uterine artery (or a good indication of volume via some indirect parameters) is paramount to assess adequately the vascular physiological changes that happen during pregnancy. Defective infiltration by trophoblasts into the uterine spiral arteries is a consistent finding in preeclampsia and IUGR. Thus, the spiral arteries remain physiologically un-modified, resulting in increased impedance to the uterine artery blood flow. This compromises the blood supply to the placenta, resulting in placental insufficiency, placing the mother or the fetus or both at a higher risk for poor outcome of the pregnancy. In a normal pregnancy there is progressively reducing downstream impedance and a progressively increasing blood volume in the uterine artery. On the other hand the high risk pregnancies have increased downstream impedance and a decreased blood volume in the uterine artery. Therefore Doppler waveform analysis of the blood volume, next to or replacing Doppler waveform analysis of the blood flow velocity, in the uterine artery will result in an improved detection of high risk pregnancy conditions.

Accessing the blood volume in the uterine artery by the proposed area ratio under the systolic and diastolic part (AR) of the maximum frequency envelope of a spectrogram has the advantages that this parameter can be determined using known techniques for acquiring said spectrogram, and that this parameter, like the Resistive Index (RI) and the Pulsatility Index (PI) can be determined from angle independent measurements (i.e., they do not depend on the angle of incidence between the ultrasound wave and the blood vessel).

It is noted that obtaining a direct measures of the blood volume in the uterine artery would involve an invasive procedure.

In a preferred embodiment of the method according to the invention, step of defining a systolic part and a diastolic part of the maximum frequency envelope comprises the sub-step of determining at least one peak (S) and one valley (D) in the maximum frequency envelope, said peak (S) corresponding to a peak systolic phase in a heart cycle and said valley (D) corresponding to an end diastolic phase in the heart cycle.

From the determined peak(s) and valley(s) in the maximum frequency envelope the systolic and a diastolic part(s) can easily be identified.

The determined area ratio under the systolic part and the diastolic part (AR) may be presented, for example on a user interface, to a user such as, for example, a medical doctor. From this presented value the user then classifies the risk of the pregnancy.

In a further preferred embodiment of the method according to the invention, the method further comprises the step of classifying the acquiring ultrasound Doppler signals as abnormal when said area ratio (AR) is greater than a predetermined threshold. This embodiment may be especially advantages when applied in an apparatus for routinely monitoring pregnancies where minimal user interaction is required (such as the one described in U.S. Patent Application 61/425866 of 22 Dec. 2010).

The predetermined threshold value may be determined by clinical studies. A threshold value of 0.60 is proposed. This value was determined in a study by the inventors.

In accordance with a further method according to the invention, the method comprises the steps of:

i) acquiring ultrasound Doppler signals from the uterine artery;
ii) generating a spectrogram from the acquired ultrasound Doppler signals and determining the maximum frequency envelope of said spectrogram;
iii) determining at least one peak (S) and one valley (D) in the maximum frequency envelope, said peak (S) corresponding to a peak systolic phase in a heart cycle and said valley (D) corresponding to an end diastolic phase in a the hart cycle;
iv) determining at least one of the Resistive Index (RI), the Pulsatility Index (PI) and the Peak Systolic Velocity/End Diastolic Velocity ratio (S/D-ratio);
v) classifying the acquiring ultrasound Doppler signals as normal or abnormal based on said least at least one determined Resistive Index (RI), Pulsatility Index (PI) or Peak Systolic Velocity/End Diastolic Velocity ratio (S/D-ratio); and when the acquiring ultrasound Doppler signals as classified as normal
vi) defining a systolic part and a diastolic part of the maximum frequency envelope and calculating an area ratio under said systolic part and said diastolic part (AR); and
vii) classifying the acquiring ultrasound Doppler signals as abnormal when said area ratio (AR) is greater than a predetermined threshold.

Step i) is a standard procedure using a regular ultrasound machine for acquiring ultrasound Doppler signals. Such a regular ultrasound machine may be an imaging device for producing ultrasound Doppler images or an ultrasound pregnancy monitoring device not capable of producing ultrasound images itself.

Steps ii) and iii) are well known steps which are, for example, part of a decision support package for obstetrics specific ultrasound Doppler velocimetry to identify abnormal pregnancies. In step iv) one or more of the blood velocity related parameters used in current clinical practice are determined in order to classify normal versus abnormal pregnancies (in step v)). It is noted that in the aforementioned U.S. Patent Application 61/425866 of 22 Dec. 2010 a device is described for assessing normality of blood flow by utilizing both the parameters PI and RI.

If, from these velocity related parameters, the pregnancy appears to be normal, further analysis steps are performed in order to improve the sensitivity and to identify those at-risk pregnancies which are missed in step v) due to less sensitivity of the current clinical parameters, like RI and PI. The blood volume related parameter AR (area ratio under the systolic part and diastolic part) is determined (step vi)) and its determined value is compared to a predetermined threshold value (step vii)). The predetermined threshold value may be determined by clinical studies or from clinical experience. A threshold value of 0.60 is proposed. This value was determined in a study by the inventors.

It is noted that alternatively step vi) and vii), in which the blood volume related parameter AR is determined, may always be executed. In this way the blood volume related parameter AR is always available next to the blood flow velocity related parameters for accessing the risks of a pregnancy.

In accordance with a system according to the invention, the system comprises means for executing the above described methods.

Such a system according to the invention may be part of a lager system, such as, for example, a diagnostic ultrasound apparatus capable of producing ultrasound images, a pregnancy monitoring apparatus or an automated clinical decision support system.

The system according to the invention may, for example, be implemented by a general purpose processor on which the appropriate software is loaded or by special purpose hardware, such as one or more integrated circuits, implementing the functions of the methods.

In accordance with a software product according to the invention, the software, when loaded on a processor, executes the steps according to the methods as claimed thereby implementing the functions of the methods.

### DESCRIPTION OF THE DRAWINGS

The above and other objects and features of the present invention will become more apparent from the following detailed description considered in connection with the accompanying drawings, in which:

FIG. 1A and 1B are graphs showing a maximum frequency envelope of a spectrogram from acquired ultrasound Doppler signals;

FIG. 2 is a graphs showing a maximum frequency envelope of a spectrogram which is divided in a systolic fraction and a diastolic fraction;

FIG. 3 and 4 show flowcharts of the steps according to embodiments of a method according to the invention.

The same reference numerals are used to denote similar parts throughout the figures.

### DETAILED DESCRIPTION

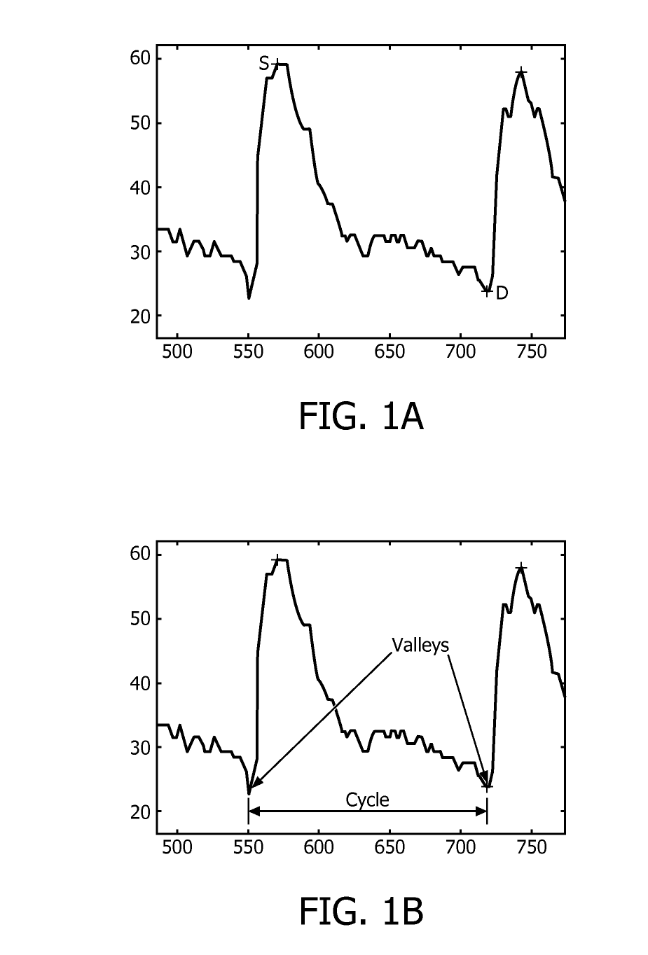

FIG. 1A and 1B show a maximum frequency envelope of a spectrogram from acquired ultrasound Doppler signals. FIG. 1A shows a peak (S) corresponding to the blood flow velocity during the peak systolic phase of a heart cycle and a valley (D) corresponding to the blood flow velocity during the end diastolic phase of a heart cycle. FIG. 1B shows a time distance A between two consecutive valleys. This distance A corresponds to the duration of a single heart cycle. It is noted that the time distance A can alternatively be determined between two consecutive peaks. The blood flow velocity related parameters can be determined from peak (S), valley (d) and time distance A.

The Peak Systolic Velocity/End Diastolic Velocity ratio (S/D-ratio) can be calculated from:

S/D-ratio=S/D;

the Pulsatility Index (PI) can be calculated by:

PI=(S−D)/A;

and the Resistive Index (RI) can be calculated by:

RI=(S−D)/S.

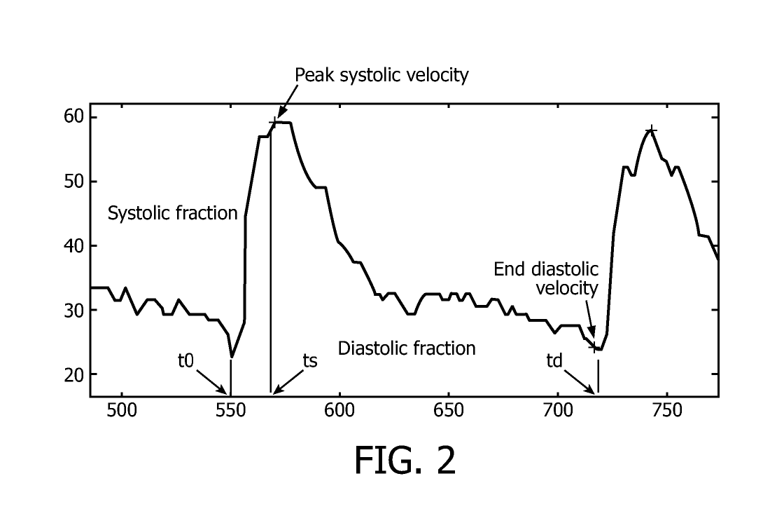

FIG. 2 shows a similar maximum frequency envelope of a spectrogram from acquired ultrasound Doppler signals. In an embodiment of the method according to the invention the area ratio under the systolic part and the diastolic part (AR) of the maximum frequency envelope is determined by defining the systolic part and the diastolic part using the peak(s) and valley(s) in this graph.

The time t₀ corresponding to a valley (i.e. the lowest velocity) in the graph is taken as the starting point of the systolic part while the time t_{S} corresponding to a peak (i.e. the highest velocity) is taken as the ending point of the systolic part. For this systolic part the Area Under the Curve AUC_{Systole} is determined by calculating the area under the maximum frequency envelope from time t₀ to time t_{S}. The time t_{D} corresponding to the next valley in the graph is taken as the ending point of the diastolic part while the time t_{S} is taken as the starting point of the diastolic part. For diastolic part the Area Under the Curve AUC_{Diastole} is determined by calculating the area under the maximum frequency envelope from time t_{S} to time t_{D}. The area ratio (AR) is now determined by:

Area ratio (AR)=AUC_{Systole}/AUC_{Diastole}.

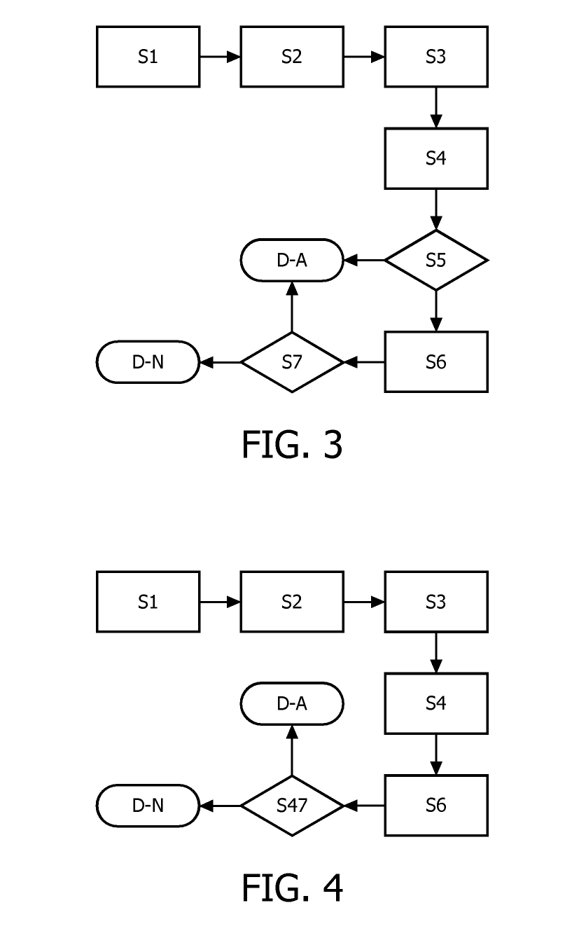

In FIG. 3 shows a flowchart of the steps according to an embodiment of a method according to the invention is shown.

In step S1 ultrasound Doppler signals resulting from the uterine artery are acquired.

In a diagnostic ultrasound apparatus these ultrasound Doppler signals are acquired by manually placing the ultrasound probe and scanning the uterine artery in a convensional way. This scanning may be done in a semi-automated fashion as is done by the pregnancy monitoring apparatus described in the aforementioned U.S. Patent Application 61/425866 of 22 Dec. 2010. In a clinical decision support system these ultrasound Doppler signals may be acquired from a storage device, such as for example a computer memory, a harddisk drive, a network Hospital Information System, or the like. The ultrasound Doppler signals are then pre-acquired by a conventional diagnostic ultrasound apparatus and stored in the storage device for retrieval by the clinical decision support system.

From these acquired ultrasound Doppler signals a spectrogram and the maximum frequency envelope of said spectrogram is generated in step S2 using conventional and well know techniques.

Next at least one peak (S) and one valley (D) in this maximum frequency envelope are determined in step S3. A peak (S) corresponds the maximum blood velocity during a systolic phase in a heart cycle and a valley (D) corresponds to the minimal blood velocity during a diastolic phase in a the hart cycle.

In step S4 the Resistive Index (RI) and the Pulsatility Index (PI) are determined as described above with reference to FIG. 1. It is noted that the Peak Systolic Velocity/End Diastolic Velocity ratio (S/D-ratio), or any other flow velocity related parameter, may be added to, or replace any of, the parameters RI and PI.

In step S5 it is determined whether the acquired ultrasound Doppler signals, and thereby the utero-placental circulation, are to be classified as abnormal (D-A) based on the parameter values determined in step S4. The way the acquired ultrasound Doppler signals are classified from the values for the parameters RI and PI is a well established clinical practice.

When the acquired ultrasound Doppler signals are not classified as abnormal in step S5 the method continues to step S6 in which a systolic part and a diastolic part of the maximum frequency envelope are determined and the area ratio under said systolic part and said diastolic part (AR) is calculated as describe above with reference to FIG. 2.

In step S7 it is determined whether the acquired ultrasound Doppler signals, and thereby the utero-placental circulation, are to be classified as abnormal (D-A) or normal (D-N) based on the area ratio calculated in step S6. According to an embodiment of the method the acquiring ultrasound Doppler signals are classified as abnormal when the area ratio (AR) is greater than a predetermined threshold.

The predetermined threshold value may be determined by clinical studies. A threshold value of 0.60 is proposed. This value was determined in a study by the inventors. The results of this study are shown in the table below:

| Threshold value | Accuracy | Sensitivity | Specificity |
|---|---|---|---|
| 0.4 | 57.1% | 100% | 13.3% |
| 0.5 | 85.7% | 100% | 71.4% |
| 0.6 | 92.9% | 100% | 85.7% |
| 0.7 | 92.9% | 85.7% | 100% |
| 0.8 | 85.7% | 71.4% | 100% |
| 0.9 | 78.6% | 57.1% | 100% |
| 1.0 | 57.1% | 14.3% | 100% |

In this respect the accuracy is defined as the number of true positives and the number of true negatives over the total number of samples, the sensitivity is defined as the ratio of the number of true positives over the sum of the number of true positives and false negatives, and the specificity is defined as the ratio of the number of true negatives over the sum of the number of true negatives and false positives. For the proposed threshold value of 0.6 both the accuracy and the sensitivity are at their maximum value. When it is desired that both the accuracy and the specificity are at their maximum value (at the cost of a reduced sensitivity) a threshold value of 0.7 may alternatively be selected.

In the embodiment of the method according to the invention described above with reference to FIG. 3 the method steps are terminated when in step S5 it is determined that the acquired ultrasound Doppler signals, and thereby the utero-placental circulation, are to be classified as abnormal (D-A). Only when the acquired ultrasound Doppler signals are not to be classified as abnormal in step S5, the method continues to determine the area ratio (AR) in order to improve the sensitivity of the method for identifying high risk pregnancies by reducing the number of false negatives. In FIG. 4 a flowchart of the steps according to an alternative embodiment is shown. In this embodiment the area ratio (AR) is always determined in step S6 next to the parameters determined in step 4. In step S47 it is now determined whether the acquired ultrasound Doppler signals, and thereby the utero-placental circulation, are to be classified as abnormal (D-A) or normal (D-N) based on the parameters determined in step S4 as well as on the area ratio (AR) calculated in step S6. This has the advantage that the classification is based on information obtained from Doppler waveform analysis of both blood flow velocity and blood volume in the uterine artery.

1. Method for determining at least one parameter that is suitable as a basis for identifying high risk pregnancies, wherein the method comprises the steps of:
a) acquiring ultrasound Doppler signals reflected from the uterine artery;
b) generating a spectrogram from the acquired ultrasound Doppler signals and determining the maximum frequency envelope of said spectrogram;
c) defining (i) a systolic part which starts at a valley and ends at a peak in the maximum frequency envelope and (ii) a diastolic part of the maximum frequency envelope which starts at the peak and ends at a subsequent valley in the maximum frequency envelope and calculating an area ratio (AR) by dividing an area under the maximum frequency envelope in said systolic part by an area under the maximum frequency envelope in said diastolic part.

2. Method according to claim 1 in which step c) comprise determining at least one peak and one valley in the maximum frequency envelope, said peak corresponding to a peak systolic phase in a heart cycle and said valley corresponding to an end diastolic phase in the heart cycle.

3. Method according to claim 1, further comprising the step of:
presenting said area ratio to a user.

4-7. (canceled)

8. A system for identifying high risk pregnancies wherein said system comprises:
a diagnostic ultrasound apparatus for acquiring ultrasound Doppler signals reflected from the uterine artery; and
a processor which is configured to carry out the steps of:
generating a spectrogram from the acquired ultrasound Doppler signals;
determining the maximum frequency envelope of said spectrogram;
defining a (i) systolic part which starts at a valley and ends at a peak in the maximum frequency envelope and (ii) a diastolic part which starts at the peak and ends at a subsequent valley in the maximum frequency envelope; and
calculating an area ratio by dividing an area under the maximum frequency envelope in said systolic part by an area under the maximum frequency envelope in said diastolic part.

9. (canceled)

10. Method according to claim 2, wherein step c) further comprises determining at least one of a Resistive Index, a Pulsatility Index and a Peak Systolic Velocity/End Diastolic Velocity ratio.

11. The system according to claim 5, wherein the processor is further configured to determine at least one peak and one valley in the maximum frequency envelope, said peak corresponding to a peak systolic phase in a heart cycle and said valley corresponding to an end diastolic phase in a the heart cycle.

12. The system according to claim 5, further comprising means for presenting said area ratio to a user.

13. The system according to claim 5, wherein the processor is further configured to classify the acquired ultrasound Doppler signals as abnormal when said area ratio is greater than a predetermined threshold.

14. The system according to claim 8, wherein the predetermined threshold value is 0.60.

15. The system according to claim 6, wherein the processor is further configured to determine at least one of a Resistive Index, a Pulsatility Index and a Peak Systolic Velocity/End Diastolic Velocity ratio.
